(19) United States
(12) Patent Application Publication
(10) Pub. No.: US 2006/0069251 A1
(43) Pub. Date: Mar. 30, 2006

(54) Methods for producing norgalanthamine, as well as isomers, salts and hydrates thereof

(76) Inventors: Ulrich Jordis, Wien (AT); Matthias Treu, Wien (AT); Manfred Hirnschall, Wien (AT); Johannes Frohlich, Dornbach/Wienerwald (AT); Laszlo Crollner, Ebenfurth (AT); Beate Kalz, Steinbrunn (AT); Thomas Kalz, Steinbrunn (AT); Peter Kuhnhackl, Wien (AT)

Correspondence Address: POPOVICH, WILES & O'CONNELL, PA;650 THIRD AVENUE SOUTH, SUITE 600, MINNEAPOLIS, MN, 55402, US

(21) Appl. No.: 10/508,867

(22) PCT Filed: Jan. 9, 2003
(86) PCT No.: PCT/AT03/00007

§ 371 Date: Oct. 26, 2005

(30) Foreign Application Priority Data
Mar. 25, 2002 (AT) A 463/2002

(51) Int. Cl. C07D 491/02

(52) U.S. Cl. 540/581

(57) ABSTRACT

The invention relates to processes, which can be performed on an industrial scale, for the production of norgalanthamine, norgalanthamine derivatives and isomers thereof of general formula (A) as well as salts or hydrates thereof with general formula (B). As processes for the production of compounds with general formula (A) or (B), an oxidative demethylation and a catalytic demethylation of the corresponding galanthamine compounds are indicated.

The invention relates to a process for the production of norgalanthamine, norgalanthamine derivatives [=(4aS,6R,8aS)-4a,5,9,10,11,12-hexahydro-3-methoxy-6H-benzofuro[3a,3,2-ef][2]benzazepin-6-ol, 10-de(s)methylgalanthamine, N-demethylgalanthamine, N-norgalanthamine] and isomers thereof with general formula A 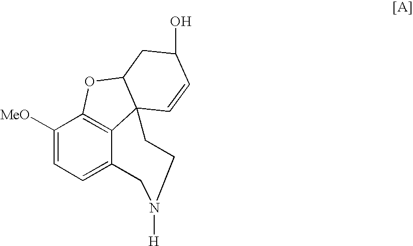
as well as salts and hydrates thereof with general formula B

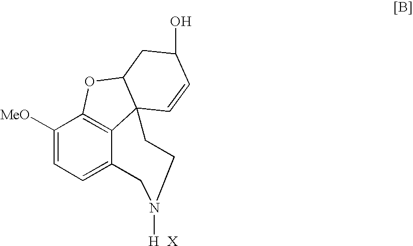

The compounds, which fall under general formula (A) or (B), are key intermediate products for the production of synthetic galanthamine derivatives, which are used for, i.a., treatment of diseases of the central nervous system (CNS), such as Alzheimer's disease.

Compounds of general formulas (A) and (B) are known and can be obtained from, for example, natural substances, see:

Sener, Bilge; Konukol, Sakine; Kruk, Cornelis; Pandit, Upendra K. Alkaloids from Amaryllidaceae. III. Alkaloids from the Bulbs of Pancratium Maritimum. Nat. Prod. Sci. 1998, 4, 148-152

Eichhorn, Jorg; Takada, Takeshi; Kita, Yasuyuki; Zenk, Meinhart H. Biosynthesis of the Amaryllidaceae Alkaloid Galanthamine. Phytochemistry 1998, 49, 1037-1047

Almanza, Giovanna R.; Fernandez, Juan M.; Wakori, Edith W. T.; Viladomat, Francesc; Codina, Carles; Bastida, Journe. Alkaloids from Narcissus cv. Salome. Phytochemistry 1996, 43, 1375-1378

Latvala, Anita; Oenuer, Mustafa A.; Goezler, Tekant; Linden, Anthony; Kivcak, Bijen; Hesse, Manfred. Alkaloids of Galanthus elwesii. Phytochemistry 1995, 39, 1229-1240.

Kreh, Mirko; Matusch, Rudolf. O-Methyloduline and N-Demethylmasonine, Alkaloids from Narcissus Pseudonarcissus. Phytochemistry 1995, 38, 1533-1535

Bastida, Jaume; Viladomat, Francesc; Bergonon, Salvador; Fernandez, Juan Marcos; Codina, Carles; Rubiralta, Marlo; Quirion, Jean Charles. Narcissus Alkaloids. Part 19. Alkaloids from Narcissus leonensis. Phytochemistry 1993, 34, 1656-1658

Weniger, B.; Italiano, L.; Beck, J.-P.; Bastida, J.; Bergonon, S.; Codina, C.; Lobstein, A.; Anton, R. Cytotoxic Activity of Amaryllidaceae Alkaloids. Planta Med. 1995, 61, 77-79

Bastida, J.; Viladomat, F.; Llabres, J. M.; Quiroga, S.; Codina, C.; Rubiralta, M. Narcissus Alkaloids. Part IX. Narcissus Nivalis: A New Source of Galanthamine. Planta Med. 1990, 56, 123-124

Kihara, Masaru; Kolke, Tomomi; Imakura, Yasuhiro; Kida, Kiyoshi; Shingu, Tetsuro; Kobayashi, Shig-atu. Alkaloidal Constituents of Hymenocallis Rotata Herb. (Amaryllidaceae). Chem. Pharm. Bull. 1987, 35, 1070-1075

Kobayashi, Shigeru; Ishikawa, Hideki; Kihara, Masaru; Shing, Tetsuro; Uyeo, Shojiro. Isolation of N-Demethylgalanthamine from the Bulbs of Crinum Asiaticum L. Var. Japanicum Baker (Amaryllidaceae). Chem. Pharm. Bull. 1976, 24, 2553-25555

In addition, it is known to produce compounds of general formula A or B synthetically by demethylation of galanthamine.

A two-stage demethylation of galanthamine by degradation of N-oxide is thus described in WO-A1-9703987.

In addition, a two-stage demethylation of galanthamine by reaction with carboxylic acid derivatives is described in Tetrahedron Lett. 1997, 38, 5151, Mary, Aude; Renko, Dolor Zafiarisoa; Guillou, Catherine; Thal, Claude “Selective N-Demethylation of Galanthamine to Norgalanthamine Via a Non-Classical Polonovski Reaction.”

The two-stage demethylation of galanthamine by reaction with azodicarboxylic acid esters and subsequent saponification is known a priori from U.S. Pat. No. 5,958,903.

In addition, the N-demethylation represents a standard reaction of alkaloid chemistry, whereby in particular N-dealkylations with the following reagents are known:
BrCN/Zn

Seiler, Max. P.; Hagenbach, Alexander; Wuethrich, Hans-Juerg; Markstein, Rudolf. J. Med. Chem. 1991, 34, 303-307
Chloroethyl chloroformate

Ghosh, Debasis; Snyder, Scott E.; Watta, Val J.; Mailman, Richard B.; Nichols, David E. J. Med. Chem. 1996, 39, 549-555
NaOCl, phase transfer catalysis, then NaOH

Robson, Claire; Meek, Michelle A.; Grunwaldt, Jan-Dierk; Lambert, Peter A.; Queener, Sherry F. J. Med. Chem. 1997, 40, 3040-3048
N-Iodosuccinimide

Stenmark, Heather G.; Brazzale, Antony; Ma, Zhenkun, J. Org. Chem. 2000, 65, 3875-3876
Methyl chloroformate

Kim, J. C. Org. Prep. Proced. Int. 1977, 9. 1
Palladium on activated carbon/O₂

Chaudhuri, Naba K.; Servando, Ofella; Markus, Bohdan; Galynker, Igor; Sung, Ming-Sang. J. Indian Chem. Soc. 1985, 62, 899-903
Ethyl chloroformate

Humber, L. G.; Charest, M. P.; Herr, F. J. Med. Chem. 1971, 14, 982
Pyridine hydrochloride

Radl, Stanislav, Pyridine Hydrochloride in Organic Synthesis. Janssen Chim. Acta 1989, 7, 12-17
Numerous chloroformates

Olafson, R. A.; Martz, Jonathan T.; Senet, Jean Pierre; Piteau, Marc; Malfroot, Thierry. A New Reagent for the Selective, High-Yield N-Dealkylation of Tertiary Amines: Improved Syntheses of Naltrexone and Nalbuphine. J. Org. Chem. 1984, 49, 2081-2082
BrCN/hydrolysis

Lee, Shoel Sheng; Liu, Yi Chu; Chang, Shu Hwei; Chen, Chung Hsiung. N-Demethylation Studies of Pavine Alkaloids. Heterocycles 1993, 36, 1971-1974
Fe/Fe²⁺/O₂

Sparfel, Daniel; Baranne-Lafont, Joele: Nguyen Kim Cuong; Capdevielle, Patrice; Maurny, Michel. II. Catalytic Oxidation of 2-(Aminomethyl)phenols with the System Ferrous Chloride-Iron-Oxygen. Tetrahedron 1990, 46, 803-814

These known processes have the drawback that they can be quite difficult to do on an industrial scale because of their low yields, the relatively expensive starting products as well as because of the reaction conditions that are occasionally hazardous.

The object of this invention is therefore to provide a process for the production of norgalanthamine, norgalanthamine derivatives as well as isomers thereof, which can also be implemented economically on an industrial scale. At the same time, however, the end products of the process are also to be produced with high purity.

The invention relates to the production of norgalanthamine, norgalanthamine derivatives and isomers thereof with general formula A 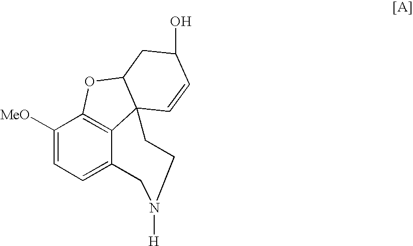
as well as salts and hydrates thereof with general formula B 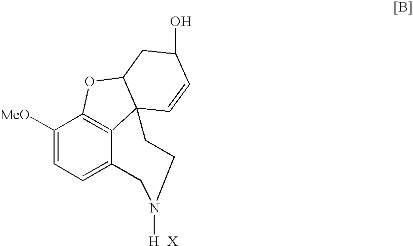
by demethylation of the corresponding galanthamine compounds with general formula (I)

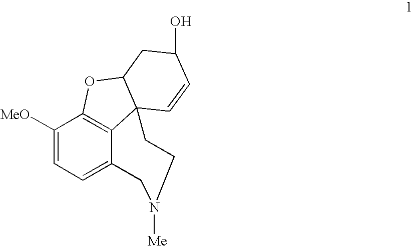

An advantageous process variant according to the invention can be explained based on the synthesis diagram below:

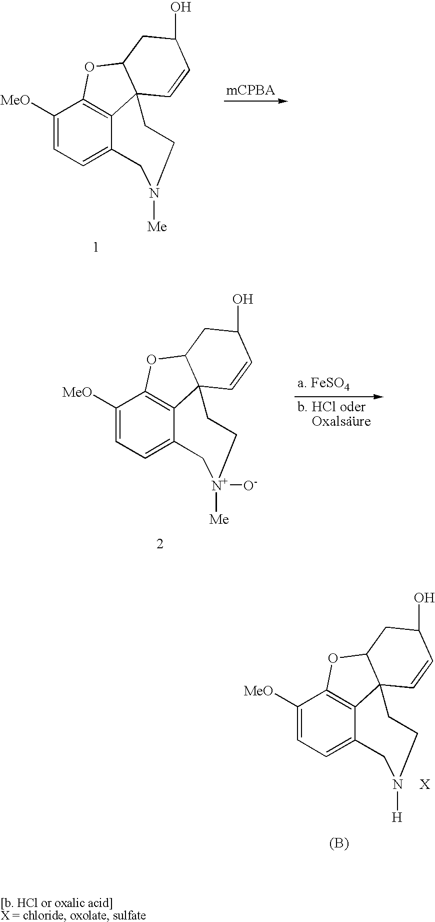

In this case, the respective galanthamine compounds with general formula (I) are reacted by oxidation in the intermediate product with general formula (2), the so-called N-oxide intermediate product.

As oxidizing agents, for example, hydrogen peroxide or substituted perbenozic acids are suitable. M-Chloroperoxybenzoic acid (mCPBA) is especially preferred.

Then, the norgalanthamine salt that can be easily separated is produced according to general formula (B) for example as chloride, oxalate or sulfate, and is separated from the reaction mixture by precipitation. The separated salt already has a high HPLC purity but can be purified in addition by further recrystallization. As a whole, the process is also suitable for implementation on an industrial scale, since a purification step using salt precipitation represents a relatively small expense even on a large scale.

Advantageous process variants according to the invention are explained in more detail based on the following exemplary embodiments:

### EXAMPLE 1

### Production of Norgalanthamine Hydrochloride (B, X═HCl)

2.10 kg (7.3 mol) of (−)-galanthamine is dissolved in 12.6 1 of chloroform while being stirred. In this solution, 1.93 kg (8.5 mol) of m-chloroperoxybenzoic acid (mCPBA) (75.6%) is added in portions during a period of 30-120 minutes such that the temperature of the solution is between 15° C. and 50° C. After one to two hours of stirring without temperature adjustment, the reaction mixture is cooled to 0-10° C. A solution of 0.20 kg (0.73 mol) of iron(II) sulfate heptahydrate in 7.30 l of water is now added in drops during a period of 40-50 minutes, such that the temperature of the reaction mixture remains between 0° C. and 10° C. After the addition is completed, the reaction mixture is allowed to stir for 10 more minutes at 0-10° C. After 1.05 kg of concentrated hydrochloric acid is added, 10.0 l of chloroform is then distilled off in a vacuum. The reaction mixture is extracted three times with 9.40 l each of MTBE at 30° C. to 40° C. to remove the benzoic acid. The aqueous phase is stirred for 5 hours to 24 hours at 0° C. to 10° C. The precipitate that is produced is suctioned off, washed with 2.0 l of MTBE and dried at 50° C. in a vacuum.

Yield: 1.87 kg (80.5% of theory)

HPLC purity 95.4%

Melting point 173-180° C.: ¹H NMR (DMSO-d₆): δ 9.70 (b, 1.5 H), 6.92-6.79 (m, 2H), 6.15 (d, 1H), 5.87 (m, 1H), 4.50 (m, 1H), 4.48-4.03 (m, 5H), 3.75 (s, 3H), 2.50-1.80 (m, 5H); ¹³C NMR (DMSO-d₆): δ 147.36 s, 145.37 s, 133.67 s, 130.30 d, 126.55 d, 123.33 s, 122.76 d, 112.69 d, 87.24 d, 60.33 d, 56.54 q, 49.97 t, 48.03 s, 45.14 t, 35.08 t, 31.63 t.

### EXAMPLE 2

### Production of Norgalanthamine Oxalate (B, X=Oxalic Acid)

If oxalic acid-dihydrate is used instead of HCl, as in Example 1, the oxalate salt of the norgalanathamine that is formed can be isolated.

Yield: 75% of theory

HPLC purity 96%

Melting point 233-235° C.; ¹H NMR (DMSO-d₆): 6 6.85-6.75 (m, 2H), 6.11 (d, J=10.31 Hz, 1H), 5.86 (dd, J=10.31, 4.4 Hz, 1H), 4.54 (b, 1H), 4.47 (d, J=15.7 Hz, 1H), 4.24 (d, J=15.1 Hz, 1H), 4.09 (b, 1H), 3.79 (s, 3H), 3.55-3.40 (m, 2H), 2.54-2.46 (m, 1H), 2.35-2.20 (m, 1H), 2.16-1.82 (m, 3H); ¹³C NMR (DMSO-d₆): δ 164.9 (s), 146.5 (s,), 144.5 (s), 132.9 (s), 129.5 (d), 125.7 (d), 122.8 (s), 121.7 (d), 111.7 (d), 86.4 (d), 59.6 (d), 55.7 (q), 49.4 (t), 47.2 (s), 44.4 (t), 34.4 (t), 30.8 (t); Anal. cld. for C₁₆H₁₉NO₃.C₂H₂O₄.0.5 H₂O: C, 58.06; H, 5.95; N, 3.76. Fnd: C, 57.91; H, 5.88; N, 3.69.

### EXAMPLE 3

### Purification of Norgalanthamine-Hydrochloride (B, X=HCl)

5.20 kg (16.8 mol) of (−)-norgalanthamine-HCl is taken up as a crude product in 10.4 l of water and dissolved at reflux temperature while being stirred. After 10-15 minutes of stirring at reflux temperature, it was cooled to 0-10° C. and stirred for 1 to 24 hours at this temperature. The precipitate is suctioned off and washed with 1.0 l of water and dried at 50° C. for 80-120 hours in a vacuum.

Yield: 3.96 kg (76.2% of theory)

HPLC purity 98.9%

Below, a comparison is made with the closest prior art, which is the teaching according to WO-A1-9703987.

1) Production of N-Oxide Intermediate Compound (2):

In WO-A1-9703987, the N-oxide formation is mentioned as a standard process. Without detailed information, it should be assumed that this intermediate product is produced and then—as depicted below—is subjected to an expensive purification.

2) Production of Compound B by Means of FeSO₄ Reduction from (2):

| Parameters | WO-A1-9703987 | Sanochemia Process |
|---|---|---|
| Batch Size | 1.73 g | 2.1 kg |
| Amount of | Approximately 60x | Approximately 10x |
| Solvent | | |
| Eq. Iron | 2 | 0.1 |
| Sulfate | | |
| Heptahydrate | | |
| Working-up | Evaporation to the Dry | Mixing with HCl, |
| | State, Extraction Between | Concentration by |
| | Methylene Chloride and | Evaporation to 50% and |
| | NaHCO₃ Solution | Extraction Between Water |
| | | and MTBE |
| Isolation of | As a Free Base by | HCl Salt by Cooling, and |
| the Product | Concentration by | Suctioning-off of the |
| | Evaporation to the Dry | Aqueous Phase |
| | State | |
| Purification | Column Chromatography | Recrystallization |
| End Product | Free Base | HCl Salt or Oxalate Salt |

Advantages of the process according to the invention compared to the process from WO-A1-9703987:

The reaction volume is significantly less by using ⅙ of the amount of solvent in comparison to WO-A1-9703987.

By using only 5% iron sulfate heptahydrate, the reaction mixture contains considerably lower iron residues than those associated with WO-A1-9703987.

During working-up, the reaction mixture is concentrated by evaporation to 50% of the total volume. Concentration by evaporation to the dry state, as is done even twice in WO-A1-9703987, is quite difficult to do on an industrial scale.

The end product of the process with general formula (B) is precipitated from the aqueous reaction mixture, for example, as a crystalline hydrochloride salt by cooling; conversely, a “white foam” that can be quite difficult to process further is formed according to WO-A-19703987.

A purification of the end product of the process according to general formula (B) to a degree of purity of >98% is possible by simple recrystallization from water. An expensive purification of the process product by column chromatography is not necessary, however, in contrast to WO-A1-9703987.

In summary, it can be said that this process variant according to the invention, because of the low iron residues and the reduced amount of solvent, is especially suitable for implementation on an industrial scale, whereby at the same time, however, satisfactory degrees of purity are achieved.

In addition, the invention relates to a process for the production of norgalanthamine, norgalanthamine derivatives as well as isomers thereof with general formula B from the corresponding galanthamine compounds (1) by means of catalytic demethylation. In this case, the reaction mixture contains suitable solvents or solvent mixtures, which in any case contain methanol. As catalysts, transition metals, preferably palladium, are treated on a suitable support material, preferably calcium carbonate, with an oxygen-containing gas mixture, preferably air, which is saturated in solvent mixtures. In addition, in an especially advantageous way, a gas mixture that consists of oxygen in a proportion of 3 to 30% by volume and an inert gas such as nitrogen or argon can be used. Then, the catalyst is separated from the reaction mixture, and the end product of the process with general formula B is obtained in crystalline form, e.g., as an oxalate or hydrochloride salt. This process variant according to the invention has the advantage that in contrast to the known processes, the reaction sequence is accomplished in one stage, and no hazardous or toxic reagents have to be used.

This process variant according to the invention is explained in more detail based on the following reaction diagram as well as an embodiment:

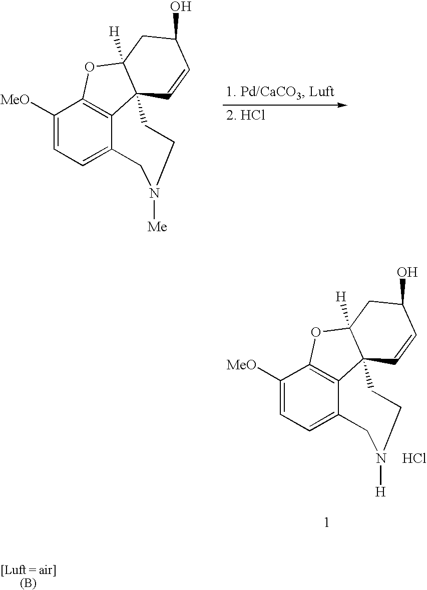

### EXAMPLE 4

### Production of (4aS,6R,8aS)-4a,5,9,10,11,12-Hexahydro-3-methoxy-6H-benzofuro[3a,3,2-ef][2]-benzazepin-6-ol, Norgalanthamine Hydrochloride (B)

10 g of galanthamine, free base, and 8 g of palladium on calcium carbonate (10%, Fluka) are introduced into an open 2 l—three-necked flask, 1 l of methanol, distilled over potassium carbonate, is added, the mixture is stirred with a magnetic stirrer and heated with an oil bath to 50° C. After seven hours of stirring, the reaction mixture is cooled, and the catalyst is allowed to settle. The solution from which catalyst is removed by decanting is concentrated by evaporation to the dry state in a Rotavapor at a bath temperature of 50° C., the residue is dissolved in 100 ml of 2N hydrochloric acid, made basic with 30% sodium hydroxide solution, and shaken out 3× with 30 ml each of chloroform. The combined organic phases are concentrated by evaporation to the dry state in a Rotavapor at a bath temperature of 50° C. The residue is taken up in 300 ml of ethyl acetate, acidified with 10% ethereal hydrochloric acid, and the precipitate is filtered off. After washing is continued once more with 20 ml of ethyl acetate, the norgalanthamine hydrochloride (B, X═HCl) is dried at 60° C. and 25 mbar.

Yield 9.4 g (95% of theory)

HPLC Purity 87.3%

Melting point 166-171° C.:

### EXAMPLE 5

### Production of (4aS,6R,8aS)-4a,5,9,10,11,12-Hexahydro-3-methoxy-6H-benzofuro-3a,3,2-ef][2]-benzazepin-6-ol, Norgalanthamine (A)

Release of norgalanthamine A from the oxalate salt or from the hydrochloride salt:

The norgalanthamine salt (oxalate B, X═HCl or hydrochloride B, X=oxalic acid) was dispersed between CH₂Cl₂ and concentrated NH₄OH solution, and the aqueous phase was exhaustively extracted with CH₂Cl₂. The combined organic phases were dried (Na₂SO₄), filtered, and solvent was removed in a rotary evaporator, by which A was obtained with yields of between 95% and 99% in the form of colorless crystals.

Melting point 145-146° C.; TLC; CHCl₃; MeOH: concentrated NH₄OH solution=90; 8.5: 1.5; ¹H NMR (CDCl₃): δ 6.65-6.52 (m, 2H), 6.06-5.92 (m, 2H), 4.57 (b, 1H) 4.15-4.08 (m, 1H), 3.95 (d, J5.7 Hz, 2H), 3.79 (s, 3H), 3.34 (ddd, J=14.6, J=3.5 Hz, J=3.5 Hz, 1H), 3.18 (ddd, J=13.2, J=11.4, J=2.6 Hz, 1H), 2.66 (ddd, J=15.7, J=1.63, J=1.63 Hz, 1H), 2.27 (b, 2H), 1.98 (ddd, J=15.7, J=5.0, J=2.4 Hz, 1H), 1.88-1.61 (m, 2H); ¹³C NMR (CDCl₃): δ 146.2 (s), 143.9 (s), 133.1 (s), 133.0 (s), 127.6 (d 127.0 (d), 120.5 (d), 111.0 (d), 88.5 (d), 61.9 (d), 55.8 (q), 53.8 (t), 48.7 (s), 47.0 (t), 40.3 (t), 29.9 (t); Anal. cld. for C₁₆H₁₉NO₃: C, 70.31; H, 7.01; N, 5.12. Fnd: C, 70.05; H, 7.01: N, 4.97.

In summary, it can be said that possible methods of production that can be implemented on an industrial scale with the process according to the invention for the production of norgalanthamine, norgalanthamine derivatives and isomers thereof, as well as salts and hydrates thereof, by N-demethylation from the corresponding galanthamine compounds are indicated. In this case, in a variant according to the invention, the corresponding galanthamine compound (1) is reacted by oxidative demethylation in an N-oxide intermediate product of general formula (2). This intermediate product with general formula (2) is then converted into salts or hydrates of norgalanthamines, norgalanthamine derivatives as well as isomers thereof with general formula (B) or into the free bases with general formula (A). Salts, such as chlorides, oxalates and sulfates, are preferably formed, which can be separated in a simple way from the reaction mixture by precipitation. If necessary, an additional purification step is possible in a simple way by recrystallization.

Another variant according to the invention for the production of norgalanthamine, norgalanthamine derivatives and isomers thereof as well as salts and hydrates thereof is indicated according to the invention by a one-stage catalytic methylation from the galanthamine compounds with general formula (B). In this case, the corresponding norgalanthamine, norgalanthamine derivative or isomer thereof is obtained in the form of its salt or as a free base in a one-stage reaction.

1. Process for the production of norgalanthamine, norgalanthamine derivatives and isomers thereof with general formula (A) 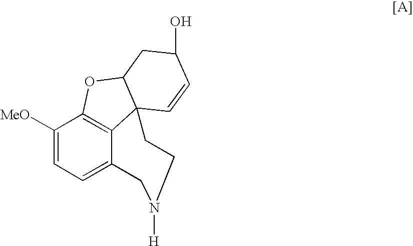
as well as salts and hydrates thereof with general formula (B) 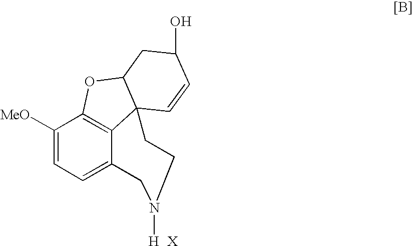
by demethylation of the corresponding galanthamine compounds with general formula (1)

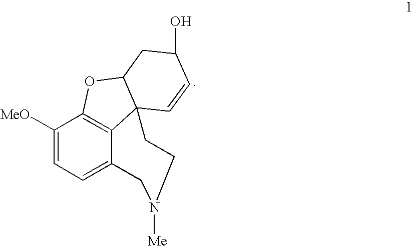

2. Process according to claim 1, characterized in that the demethylation reaction is an oxidative demethylation reaction.

3. Process according to claim 2, wherein peroxides are used as oxidizing agents.

4. Process according to claim 3, wherein substituted and/or unsubstituted perbenzoic acids, in particular m-chloroxybenzoic acid (nCPBA), are used as peroxides.

5. Process according to claim 2, wherein an N-oxide intermediate product with general formula (2) 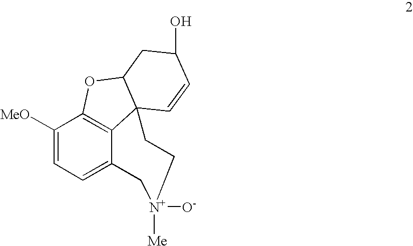
is formed by oxidative demethylation.

6. Process according to claim 5, wherein the compounds of general formula (A) or (B) are formed from the N-oxide intermediate product with general formula (2).

7. Process according to claim 6, wherein the compounds with general formula (B) are formed in the form of their salts preferably by the addition of hydrochloric acid and/or oxalic acid.

8. Process according to claim 6, wherein the compounds with general formula (B) are formed in the form of sulfates by adding iron sulfate, in particular iron sulfate heptahydrate in a 0.1 to 2.0 equimolar amount.

9. Process according to claim 7, wherein the salts of the compounds with general formula (B) are formed by recrystallization from one or more solvents.

10. Process according to claim 1, wherein the demethylation reaction is performed in the presence of a catalyst.

11. Process according to claim 10, wherein the reaction that is performed in the presence of a catalyst is a one-stage reaction.

12. Process according to claim 10, wherein palladium is used as a catalyst.

13. Process according to claim 10, wherein the catalyst is used with a support material, preferably calcium carbonate.

14. Process according to claim 11, wherein the catalytic N-methylation reaction is performed in a solvent mixture that contains at least methanol.

15. Process according to claim 10, wherein for the demethylation reaction, a mixture of oxygen, preferably 3 to 30% oxygen by volume, and an inert gas, preferably nitrogen or argon, is used.

16. Process according to claim 8, wherein the salts of the compounds with general formula (B) are formed by recrystallization from one or more solvents.

17. Process according to claim 11, wherein palladium is used as a catalyst.

18. Process according to claim 3, wherein an N-oxide intermediate product with general formula (2) 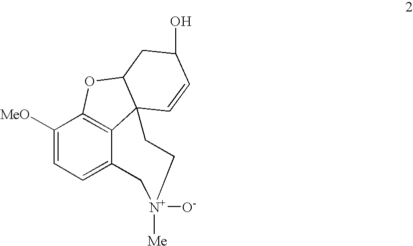
is formed by oxidative demethylation.

19. Process according to claim 4, wherein an N-oxide intermediate product with general formula (2) 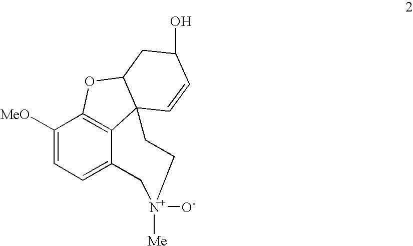
is formed by oxidative demethylation.
